(19) United States
(12) Patent Application Publication
(10) Pub. No.: US 2024/0261495 A1
(43) Pub. Date: Aug. 8, 2024

(54) Device For Administration of a Therapeutic Agent Solution and Method Comprising Same

(71) Applicants: BAXTER INTERNATIONAL INC., Deerfield, IL (US); BAXTER HEALTHCARE SA, Glattpark (Opfikon) (CH)

(72) Inventors: Michael Tung-Kiung Ling, Vernon Hills, IL (US); Benjamin Chunman Poon, Lincolnshire, IL (US)

(21) Appl. No.: 18/433,843

(22) Filed: Feb. 6, 2024

Related U.S. Application Data
(60) Provisional application No. 63/443,961, filed on Feb. 7, 2023.

(51) Int. Cl. A61M 5/14; A61K 47/06; A61K 47/36

(52) U.S. Cl. CPC A61M 5/1409; A61K 47/06; A61K 47/36; A61M 5/1414; A61M 2205/3334; A61M 2205/6009; A61M 2205/6072

(57) ABSTRACT

A device and method for parenteral administration of a solution of a therapeutic agent include a fluid reservoir having a chamber for holding fluid and an outlet port. The outlet port is fluidly coupled to a therapeutic agent conduit having a fluid inlet and a fluid outlet that define a fluid flow path. An infusion device is fluidly connected to the fluid outlet of the therapeutic agent conduit. The infusion device is capable of parenteral administration of fluid. A polymeric matrix is disposed in the therapeutic agent conduit, the polymeric matrix having a therapeutic agent dispersed therein. The therapeutic agent is at least partially dissolvable when contacted by fluid and a solution of the therapeutic agent is formed when contacted by the fluid.

### FIELD OF THE DISCLOSURE

The disclosure relates generally to therapeutic agent administration devices and more specifically to parenteral therapeutic agent administration devices and methods.

### DESCRIPTION OF THE PRIOR ART

Medication can enter the body in a number of ways. Medication may be ingested and absorbed through the stomach and intestines. Medication may be absorbed directly through the skin. Medication may be inhaled and absorbed through the lungs. Finally, medication may be injected directly into body tissues and absorbed there. Injected medication may have advantages over other methods. For example, injected medication may be absorbed into the blood stream quickly, and the medication may avoid a first pass metabolism in the liver, depending on where the medication is injected, which can preserve the efficacy of certain medications.

Medications may be injected into the body in different ways. For example, medication may be injected intramuscularly, subcutaneously, intravenously, or intradermally. Each of these injection methods have certain advantages and disadvantages, but all of these methods introduce medication directly into internal bodily tissue. Intramuscular, subcutaneous, and intradermal injections are usually referred to as “shots,” and can be used to inject medications such as vaccines, anti-inflammatory drugs, and other medications that are intended to be absorbed more slowly.

When rapid absorption and/or direct introduction of medication to the bloodstream is needed, the most common method of administering the medication is intravenously. In intravenous administration of medication, the medication is usually a liquid, or held in liquid form, such as in a solution of medication. The liquid medication is infused directly into a patient's blood stream through venous access with a needle or a cannula. Intravenous administration of medication is very effective. However, the medication must be in liquid form. Some medications that are stable in solid form may be difficult to convert to a liquid and/or may have a short shelf life in liquid form.

### SUMMARY

In a first example, a device for parenteral administration of a solution of a therapeutic agent includes a fluid reservoir comprising a chamber for holding fluid and an outlet port. The outlet port is fluidly coupled to a therapeutic agent conduit having a fluid inlet and a fluid outlet that define a fluid flow path. The fluid inlet is fluidly coupled to the outlet port of the fluid reservoir. An infusion device is fluidly connected to the fluid outlet of the therapeutic agent conduit. The infusion device is capable of parenteral administration of fluid. A polymeric matrix is disposed in the therapeutic agent conduit, the polymeric matrix having a therapeutic agent dispersed therein. The therapeutic agent is at least partially dissolvable when contacted by fluid. A solution of the therapeutic agent is formed when contacted by the fluid and is adapted for parenteral administration through the infusion device.

The above example of a parenteral therapeutic agent administration device may further include any one or more of the following optional forms.

In one optional form, the polymeric matrix comprises one or more water-insoluble polymers. In some optional forms, the water-insoluble polymer comprises one of poly(ethyl-vinyl acetate) (EVA), ultra low density polyethylene (ULDPE), linear low density polyethylene (LLDPE), high density polyethylene (HDPE), ethylene methyl acrylate (EMA), ethylene acrylic acid (EAA), ethylene ethyl acrylate (EEA), polypropylene, propylene copolymers, styrenic block copolymers, olefin block copolymers, polyurethanes, or insoluble polysaccharides, or combinations thereof.

In other optional forms, the polymeric matrix comprises a water swellable polymer, such as super-absorbent polymer. Super-absorbent polymers are cross-linked polymers that do not dissolve in water. Super-absorbent polymers generally fall in to three categories. A first category includes cross-linked polyacrylates and polyacrylamides. A second category includes cellulose or starch acrylonitrile graft copolymers. A third category includes cross-linked maleic anhydride copolymers. In some optional forms, the water swellable polymer comprises one of polyacrylate (acrylic acid and acrylamide), polyvinyl alcohol, poly(ethylene oxide), starch-acrylate copolymer, and carboxymethyl cellulose.

In other optional forms, the therapeutic agent conduit includes an outer conduit surface having a first layer comprising the polymeric matrix, an interior conduit surface and the first layer defining at least a portion of the fluid flow path.

In other optional forms, the inner conduit surface includes a second layer comprising the polymeric matrix coupled to the first layer, the second layer being disposed radially outward of the first layer. The first layer comprises the therapeutic agent at a first concentration and the second layer comprises the therapeutic agent at a second concentration.

In other optional forms, a content of the therapeutic agent in the first layer is in the range of about 5% to about 70%, based on the entire weight of the first layer.

In other optional forms, a content of the therapeutic agent in the second layer is in the range of about 5% to about 70%, based on the entire weight of the second layer.

In other optional forms, the therapeutic agent conduit further comprises a housing enclosing the polymeric matrix within the fluid flow path.

In other optional forms, the polymeric matrix comprises particles of polymeric material.

In other optional forms, the polymeric matrix comprises pellets of polymeric material.

In other optional forms, the housing further comprises an inlet and an outlet, which are arranged along the fluid flow path.

In other optional forms, the inlet comprises an inlet membrane permeable to the fluid and the outlet comprises an outlet membrane permeable to the solution. One or both of the inlet membrane and the outlet membrane may be impermeable to particulate polymeric matrix or other suspended solids.

In other optional forms, the polymer matrix has a porosity of greater than 5%.

In other optional forms, a roller clamp is disposed on the therapeutic agent conduit.

In other optional forms, the polymeric matrix is disposed in the therapeutic agent conduit upstream of the roller clamp.

In other optional forms, the polymeric matrix is disposed in the therapeutic agent conduit downstream of the roller clamp.

In other optional forms, a piggyback junction is disposed along the therapeutic agent conduit.

In other optional forms, the polymeric matrix is disposed in the therapeutic agent conduit upstream of the piggyback junction.

In other optional forms, the polymeric matrix is disposed in the therapeutic agent conduit downstream of the piggyback junction.

In other optional forms, the infusion device comprises one of a cannula or a needle.

In other optional forms, an infusion pump is operably connected to the fluid reservoir.

In another example, a method for parenterally administering a solution includes allowing a fluid from a fluid reservoir to flow through a fluid flow path in a therapeutic conduit. Some therapeutic agent is dissolved by the fluid from a polymeric matrix and a solution of therapeutic agent is created. The solution of therapeutic agent is delivered to an infusion device at a predetermined concentration.

The above method for parenterally administering a therapeutic agent solution may further include one or more of the following optional forms.

In one optional form, the predetermined concentration is controlled by controlling the flow rate of fluid through the fluid flow path.

In another optional form, the flow rate of fluid is controlled by adjusting a roller clamp.

In another optional form, the flow rate of fluid is controlled by adjusting an infusion pump.

In another optional form, the solution of therapeutic agent is administered intravenously to a patient.

### BRIEF DESCRIPTION OF THE DRAWINGS

FIG. 1 is a schematic drawing of a device for parenteral administration of a solution of therapeutic agent;

FIG. 2A is a perspective view of one embodiment of a therapeutic agent conduit of the device of FIG. 1;

FIG. 2B is a cross-sectional view of the therapeutic agent conduit of FIG. 2A; and

FIG. 3 is a schematic view of another embodiment of a therapeutic agent conduit of the device of FIG. 1.

### DETAILED DESCRIPTION

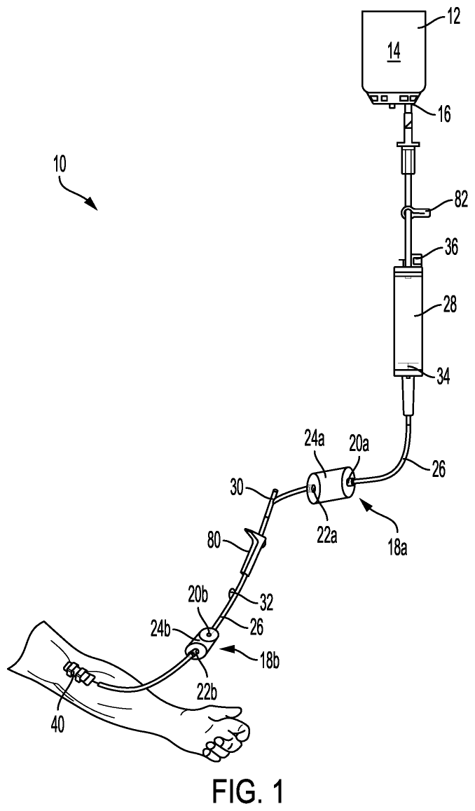

Turning now to the FIG. 1, a device 10 for parenteral administration of a solution of a therapeutic agent is illustrated. The device 10 in the illustrated embodiment comprises an intravenous infusion assembly. In other embodiments, the device 10 may comprise other types of infusion or injection assemblies, such as Intramuscular, subcutaneous, and intradermal injection assemblies. In yet other embodiments, the device 10 may comprise any type of medical device that administers a fluid medication.

The device 10 includes a fluid reservoir 12, such as an IV bag in the illustrated embodiment. In other embodiments, the fluid reservoir 12 may comprise an infusion pump (not shown), or other type of fluid reservoir. The fluid reservoir 12 comprises a chamber 14 for holding fluid. Typically, the fluid is a carrier solution, for example, a saline or other electrolyte solution (salt concentrations may vary), or a dextrose solution (again, concentrations may vary), but in some embodiments other types of fluids, such as water for injection may be used. The fluid reservoir 12 also comprises an outlet port 16. In other embodiments, the fluid reservoir 12 may comprise more than one outlet port 16. The outlet port is fluidly coupled to a therapeutic agent conduit 18a, 18b, which can be directly incorporated into a section of IV tubing (i.e., conduit 18a, 18b are integrally formed parts of the IV tubing) or a separate chamber connected to the IV tubing. As illustrated in FIG. 1, the device 10 may comprise a plurality of therapeutic agent conduits 18a, 18b, for example, the device 10 may comprise two therapeutic agent conduits 18a, 18b, three therapeutic agent conduits 18a, 18b, four (or more) therapeutic agent conduits 18a, 18b, that deliver multiple therapeutic agents, or different concentrations of the same therapeutic agent. In the illustrated embodiment, the therapeutic agent conduits 18a, 18b are connected in series to the fluid reservoir 12. In other embodiments, the therapeutic agent conduits 18a, 18b may be connected in parallel to the fluid reservoir 12. In yet other embodiments, having three or more therapeutic agent conduits 18a, 18b, some therapeutic agent conduits 18a, 18b may be connected in series while other therapeutic agent conduits are connected in parallel. Alternatively, the device 10 may comprise a single therapeutic agent conduit 18a, 18b at any location, for example, at either location illustrated in FIG. 1. The therapeutic agent conduit 18a, 18b comprises a fluid inlet 20a, 20b and a fluid outlet 22a, 22b that define a fluid flow path 24a, 24b. The fluid inlet 20a 20b is fluidly coupled to the outlet port 16 of the fluid reservoir 12. The fluid inlet 20a, 20b may be directly fluidly coupled to the outlet port 16, or, alternatively, the fluid inlet 20a, 20b may be indirectly fluidly coupled to the outlet port 16. For example, in the embodiment illustrated in FIG. 1, the fluid inlet 20a, 20b is indirectly fluidly coupled to the outlet port 16 because other fluid carrying elements, such as a drip chamber 28, and/or a restrictive device 80, are located between the fluid inlet 20a, 20b, and the outlet port 16.

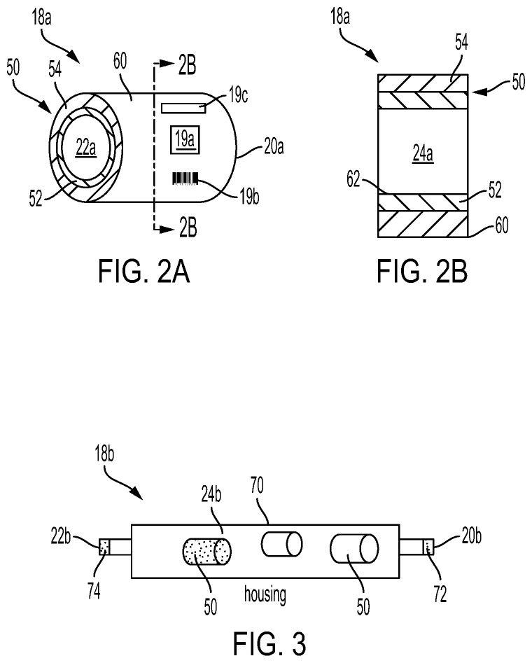

In the illustrated embodiment, the therapeutic agent conduit 18a, 18b may be indirectly fluidly connected to the fluid reservoir 12 by intervening structures normally found in IV assemblies, such as IV tubing 26, the drip chamber 28, piggyback junctions 30, and injection ports 32. The intervening structures may have features generally known in the art, such as disc valves 34 and air vents 36. While the therapeutic agent conduits 18a, 18b illustrated in FIG. 1 appear to be larger than the connecting IV tubing 26, in some embodiments, for example in the embodiment illustrated in FIGS. 2A and 2B below, a diameter of the therapeutic agent conduit 18a, 18b could be the same as connecting IV tubing 26, or even smaller than the connecting IV tubing, depending upon the needs of the system.

An infusion device 40, such as a needle or a cannula, is fluidly connected to the fluid outlet 22a, 22b, of the therapeutic agent conduit 18a, 18b, either directly or indirectly. In the embodiment illustrated in FIG. 1, the fluid outlets 22a, 22b are indirectly connected to the infusion device 40 by IV tubing 26. The infusion device 40 is capable of parenteral administration of fluid. For example, the infusion device 40 is configured to be capable of administering fluid (e.g., liquid medication) intramuscularly, subcutaneously, intravenously, and intradermally.

A polymeric matrix 50 (FIGS. 2A, 2B, and 3) is disposed in the therapeutic agent conduit 18a, 18b. While only one therapeutic agent conduit 18a is illustrated in FIGS. 2A, 2B, and 3, other therapeutic agent conduits (such as the therapeutic agent conduit 18b in FIG. 1) may have the same features described herein with reference to the therapeutic agent conduit 18a. The polymeric matrix 50 comprises a therapeutic agent dispersed therein. The therapeutic agent is at least partially dissolvable when contacted by fluid, for example fluid from the fluid reservoir 12. A solution of the therapeutic agent is formed when the polymeric matrix 50 is contacted by the fluid and the solution of therapeutic agent is adapted for parenteral administration through the infusion device 40. The polymeric matrix 50 may be formed as tubing, a film, an injection molded part, such as a pellet (which may take the form of a pill or tablet, or any other discrete shape or size), or a porous media.

Generally, the polymeric matrix 50 comprises one or more water-insoluble polymers in combination with the therapeutic agent. In some embodiments, the water-insoluble polymer comprises one of poly(ethyl-vinyl acetate) (EVA), ultra low density polyethylene (ULDPE), linear low density polyethylene (LLDPE), high density polyethylene (HDPE), ethylene methyl acrylate (EMA), ethylene acrylic acid (EAA), ethylene ethyl acrylate (EEA), polypropylene, propylene copolymers, styrenic block copolymers, olefin block copolymers, polyurethanes, or insoluble polysaccharides, or combinations thereof. In other embodiments, the polymeric matrix 50 comprises one or more water swellable polymers in combination with the therapeutic agent. Useful water-swellable materials, such as super absorbent polymers, may also comprise the foregoing polymers and are able to absorb more than 30 wt. % in pure water, preferably at least 100 wt. %, based on an initial mass. Some useful super absorbent polymers include polyacrylate (acrylic acid and acrylamide), polyvinyl alcohol, poly(ethylene oxide), starch-acrylate copolymer, and carboxymethyl cellulose.

The therapeutic agent conduit 18a, 18b may include a safety feature, such as an RFID 19a, a barcode 19b, a magnetic strip 19c, or other identifying device to ensure that the correct medication is selected and included for delivery to the patient.

Returning now to FIGS. 2A and 2B, the therapeutic agent conduit 18a may include an outer conduit surface 60 having a first layer 52 comprising the polymeric matrix 50 oriented towards the fluid flow path 24a. An interior conduit surface 62 and the first layer 52 define at least a portion of the fluid flow path 24a.

The inner conduit surface 62 may include an optional second layer 54 also comprising a polymeric matrix, which can be the same as, or different from, the polymeric matrix 50 of the first layer 52, and that is coupled to the first layer 52. In the embodiment illustrated in FIGS. 2A and 2B, the outer surface of the second layer 54 provides the outer conduit surface 60. In other embodiments, the second layer 54 may be disposed on an inside surface of another layer that forms the outer conduit surface 60. In the embodiment of FIGS. 2A and 2B, the second layer 54 is disposed radially outward of the first layer 52. The first layer 52 comprises the therapeutic agent at a first concentration and the second layer 54 comprises the therapeutic agent at a second concentration. In other embodiments, the first layer 52 may comprise a first therapeutic agent and the second layer 54 may comprise a second therapeutic agent. In yet other embodiments, more layers may be used that comprise the polymeric matrix 50 for sequenced administration of different therapeutic agents and/or of the same therapeutic agent at different concentrations. For example, three, four, five, six, or more layers may be optionally employed to accomplish sequenced administration of one or more therapeutic agents and/or of the same therapeutic agent at different concentrations. The embodiment of FIGS. 2A and 2B may be manufactured by co-extrusion, extrusion, molding, or imbibing. During the manufacturing process, the therapeutic agent may be metered into the polymeric material at a predetermined rate to achieve desired concentrations of the therapeutic agent in solution when dissolved, as described further below.

In some embodiments, a content of the therapeutic agent in the first layer 52 is in the range of about 5% to about 70%, based on the entire weight of the first layer.

In some embodiments, a content of the therapeutic agent in the second layer 54 is in the range of about 5% to about 70%, based on the entire weight of the second layer.

Turning now to FIG. 3, a second embodiment of a therapeutic agent conduit 18b is illustrated. The therapeutic agent conduit 18b may comprise a housing 70 enclosing the polymeric matrix 50 within the fluid flow path 24b. The polymeric matrix 50 may comprise discrete units or pellets of polymeric material (such as pills or tablets), or other polymeric material structures, such as sheets or fibers. The polymeric material in the embodiment of FIG. 3 may be selected from the group of polymer materials listed above with respect to FIGS. 2A and 2B.

The housing comprises the inlet 20b and the outlet 22b, which are arranged along the fluid flow path 24b. In some embodiments, the inlet 20b may comprise an inlet filter or inlet membrane 72. The inlet membrane 72 is permeable to the fluid flowing from the fluid reservoir 12. Similarly, the outlet 22b may comprise an outlet filter or an outlet membrane 74. The outlet membrane 74 is permeable to the solution formed by the fluid and the therapeutic agent. The outlet membrane 74 is impermeable to particulate polymeric matrix 50 or other suspended solids to keep the fluid flowing to the infusion device 40 particle free. In some embodiments, the polymeric matrix 50 may be incorporated into a portion of the outlet filter or membrane 74. In other embodiments, the polymeric matrix may be incorporated into a portion of the inlet filter or membrane 72. For example, in some embodiments, the polymeric matrix 50 may be incorporated into a filter media, such as a screen or fiber mesh, by coating the screen or fiber mesh with the polymeric matrix 50.

In the embodiment of FIG. 3, the polymeric matrix 50 may have a porosity of greater than 5%.

Concentration of the therapeutic agent in the solution after the fluid has passed through the fluid flow path 24a, 24b may be controlled by several factors. The factors to consider in determining the final concentration of therapeutic agent include the concentration of therapeutic agent in the polymeric matrix 50, the porosity of the polymeric matrix 50, the surface area of the polymeric matrix 50, the flow rate of fluid through the fluid flow path 24a, 24b, polymeric matrix material selection, the temperature of the fluid flowing through the fluid flow path 24a, 24b, and the solubility of the therapeutic agent. These factors may be controlled to set a final concentration of therapeutic agent in the solution delivered to the patient.

The flow rate of fluid may also further be controlled, for example, by a restrictive device 80, such as a roller clamp located along the fluid flow path 24a, 24b between the fluid reservoir 12 and the infusion device 40. For example, in some embodiments, the restrictive device 80 may be located along the IV tubing 26.

The therapeutic agent conduit 18a may be disposed upstream of the restrictive device 80 in some embodiments. In other embodiments, the therapeutic agent conduit 18b may be disposed downstream of the restrictive device 80.

In some embodiments, a temporary shutoff device 82, such as a slide clamp, may be located upstream of the therapeutic agent conduit 18a, 18b. The temporary shutoff device 82 may be used to temporarily stop the flow of fluid from the fluid reservoir 12. The temporary shutoff device 82 may be used, for example, when repositioning the infusion device 40, or when changing the therapeutic agent conduit 18a, 18b, for example when the therapeutic agent has been exhausted in one therapeutic agent conduit 18a, 18b.

In some embodiments, an additional port, such as the piggyback junction 30, may be disposed along the therapeutic agent conduit 18a, 18b, or along IV tubing 26 between therapeutic agent conduits 18a, 18b. In some embodiments, the therapeutic agent conduit 18a may be disposed upstream of the additional port. In other embodiments, the therapeutic agent conduit 18b may be disposed downstream of the additional port. In yet other embodiments another therapeutic agent conduit 18a may be located upstream of a secondary inlet of the piggyback junction 30.

In some embodiments, the therapeutic agent conduit 18a, 18b may be disposed upstream of the drip chamber 28 to remove any trapped gas in the liquid before being delivered to the patient. In other embodiments, where the therapeutic agent conduit 18a, 18b is disposed downstream of the drip chamber 28, some other gas removal device should be installed downstream of the therapeutic agent conduit 18a, 18b, to prevent trapped gas from being delivered to the patient.

In some optional embodiments, the fluid reservoir 12 may be disposed in an infusion pump (not shown), or an infusion pump may be operably connected to the fluid reservoir 12. An infusion pump may advantageously control flow rates and fluid temperatures to achieve desired concentrations of the therapeutic agent in the solution.

Generally, the device 10 for parenteral administration of a solution of a therapeutic agent described above may be used as outlined below to create and deliver a solution of therapeutic agent to a patient at a desired concentration. Fluid flows from the fluid reservoir 12 through the fluid flow path 24a, 24b in the therapeutic conduit 18a, 18b. Optional devices may be fluidly connected between the fluid reservoir 12 and the fluid flow path 24a, 24b. Some optional devices are described above, such as the IV tubing 26, the drip chamber 28, the injection port 32, and the piggyback junction 30. As fluid flows through the therapeutic agent conduit 18a, 18b, some therapeutic agent is dissolved by the fluid and a solution of therapeutic agent is created. The solution of therapeutic agent is delivered to the infusion device 40 at a predetermined concentration. The predetermined concentration of the solution may be controlled by controlling the flow rate of fluid through the fluid flow path 24a, 24b. For example, the flow rate of fluid may be controlled by adjusting the roller clamp 80.

The devices (and methods) for parenteral administration of a solution of therapeutic agent described above may be advantageously allow therapeutic agents that are otherwise unstable in liquid form (or have short shelf lives in liquid form) to be mixed immediately before introduction to the patent. Other advantages include inexpensive manufacture, less space for storage (as opposed to storing in liquid form), easily incorporated into a standard IV assembly by attaching to a luer connection, and reducing the needed for cold storage of certain liquid therapeutic agents.

While various embodiments have been described herein, it will be understood that modifications may be made thereto that are still considered within the scope of the appended claims.

1. A device for parenteral administration of a solution of a therapeutic agent, the device comprising:
a fluid reservoir comprising a chamber for holding fluid and an outlet port;
a therapeutic agent conduit having a fluid inlet and a fluid outlet that define a fluid flow path, the fluid inlet being fluidly coupled to the outlet port of the fluid reservoir; and,
an infusion device fluidly connected to the fluid outlet of the therapeutic agent conduit, the infusion device being capable of parenteral administration of fluid, and
a polymeric matrix disposed in the therapeutic agent conduit, the polymeric matrix having a therapeutic agent dispersed therein, the therapeutic agent being at least partially dissolvable when contacted by fluid, thereby forming a solution of the therapeutic agent for parenteral administration through the infusion device.

2. The device of claim 1, wherein the polymeric matrix comprises one or more water-insoluble polymers chosen from poly(ethyl-vinyl acetate) (EVA), ultra low density polyethylene (ULDPE), linear low density polyethylene (LLDPE), high density polyethylene (HDPE), ethylene methyl acrylate (EMA), ethylene acrylic acid (EAA), ethylene ethyl acrylate (EEA), polypropylene, propylene copolymers, styrenic block copolymers, olefin block copolymers, polyurethanes, and insoluble polysaccharides.

3. The device of claim 1, wherein the therapeutic agent conduit includes an outer conduit surface having a first layer comprising the polymeric matrix oriented towards the fluid flow path, an interior conduit surface and the first layer defining at least a portion of the fluid flow path.

4. The device of claim 3, wherein the outer conduit surface includes a second layer comprising the polymeric matrix coupled to the first layer, the second layer being disposed radially outward of the first layer, the first layer comprising the therapeutic agent at a first concentration and the second layer comprising the therapeutic agent at a second concentration.

5. The device of claim 3, wherein a content of the therapeutic agent in the first layer is in the range of about 5% to about 70%, based on the entire weight of the first layer.

6. The device of any claim 4, wherein a content of the therapeutic agent in the second layer is in the range of about 5% to about 70%, based on the entire weight of the second layer.

7. The device of claim 1, wherein the therapeutic agent conduit further comprises a housing enclosing the polymeric matrix within the fluid flow path.

8. The device of claim 7, wherein the polymeric matrix comprises discrete units of polymeric material.

9. The device of claim 8, wherein the discrete units of polymeric matrix comprise one of pellets, fibers, and sheets of polymeric material.

10. The device of claim 7, wherein the housing further comprises an inlet and an outlet, which are arranged along the fluid flow path.

11. The device of claim 10, wherein the inlet comprises an inlet membrane permeable to the fluid and the outlet comprises an outlet membrane permeable to the solution, at least one of the inlet membrane and the outlet membrane being impermeable to particulate polymeric matrix or other suspended solids.

12. The device of claim 7, wherein the polymeric matrix has a porosity of greater than 5%.

13. The device of claim 1, further comprising a roller clamp disposed on the therapeutic agent conduit.

14. (canceled)

15. (canceled)

16. The device of claim 1, further comprising a piggyback junction disposed along the therapeutic agent conduit.

17-20. (canceled)

21. The device of claim 1, further comprising a plurality of therapeutic agent conduits.

22. The device of claim 21, wherein at least two of the therapeutic agent conduits in the plurality of therapeutic agent conduits are fluidly connected in series.

23. The device of claim 22, wherein at least two of the therapeutic agent conduits in the plurality of therapeutic agent conduits are fluidly connected in parallel.

24. A method for parenterally administering a solution comprising a therapeutic agent to a subject, comprising:
providing a device according to claim 1; and,
allowing a fluid from the fluid reservoir to flow through the fluid flow path in the therapeutic conduit;
partially dissolving some of the therapeutic agent in the polymeric matrix, thereby creating a solution of therapeutic agent; and
delivering the solution of therapeutic agent to the infusion device at a predetermined concentration.

25. The method of claim 24, further comprising controlling the predetermined concentration by controlling the flow rate of fluid through the fluid flow path.

26. (canceled)

27. (canceled)

28. The method of claim 24, further comprising administering the solution of therapeutic agent intravenously to a patient.
